(19) United States
(12) Patent Application Publication
(10) Pub. No.: US 2005/0131435 A1
(43) Pub. Date: Jun. 16, 2005

(54) Microkeratome cutting-head for use with a single-bevel cutting-blade assembly

(76) Inventors: Peter J. Halecki, Rochester, NY (US); Michael H. Dobner, Honeoye Falls, NY (US); Craig Barrile-Josephson, Rochester, NY (US); James A. Kirch, Scottsville, NY (US)

Correspondence Address: Michael L. Smith;Bausch & Lomb Incorporated, One Bausch & Lomb Place, Rochester, NY, 14604-2701, US

(21) Appl. No.: 10/734,497

(22) Filed: Dec. 12, 2003

(51) Int. Cl. A61F 9/00

(52) U.S. Cl. 606/166

(57) ABSTRACT

A cutting-head 46 for a microkeratome includes a slot 48 for holding a single-bevel cutting-blade assembly 20. The slot 48 is oriented so that the single-bevel cutting-blade assembly 20 will be positioned to have a shear-face angle 44 and a blade-edge point 32 approximately the same as those for a multi-beveled cutting-blade assembly.

### BACKGROUND OF THE INVENTION

1. Field of the Invention

The present invention is related to microkeratomes for cutting corneal tissue of a patient's eye, including forming a corneal flap for LASIK surgery. More specifically, the present invention is related to a microkeratome cutting-head for use with a single-bevel cutting-blade assembly.

2. Description of Related Art

It is well known to use a microkeratome to cut a slice of corneal tissue from a patient's eye. Typically, the corneal tissue is removed from the patient's eye for corneal graphs or more recently, a corneal flap is created prior to a patient undergoing laser-assisted in-situ keratomileusis (LASIK) surgery.

It is important for the surgeon to know the thickness of a patient's cornea prior to forming a corneal flap and it is also important that a known or thickness of corneal flap be formed. This then allows the surgeon to safely remove the necessary amount of corneal tissue from the patient's eye to correct the patient's eyesight, and yet, leave a sufficient amount of corneal tissue to prevent post-operative complications.

Typically, microkeratomes have been designed for use with cutting-blade assemblies formed from razor blade stock. Such razor blade stock is available from companies, such as American Safety Razor. The use of such multi-beveled blades, such as shown in FIGS. 1 and 2 has been the subject of much work and development in the microkeratome arts to provide more consistency in flap thickness while also minimizing the potential for damage to the corneal flap during use of the microkeratome. Examples of some advancements include U.S. Pat. No. 6,554,847 B2, entitled Zero Compression Microkeratome Cutting Head Assembly and U.S. patent application Ser. No. 10/334,358 filed on Dec. 30, 2002, entitled Microkeratome Cutting-blade Assembly, both of which are assigned to the present assignee and incorporated herein in their entirety by reference.

From the teachings of the prior art, it is known that the corneal flap should not be compressed and that there is a preferred area relative to the cutting-head in which the cutting edge of the cutting-blade should extend.

In addition, there are new advancements in microkeratome blade assemblies. Such advancements include the use of ceramic materials for the cutting-blade instead of the commonly used razor blade stock. Some examples of ceramic blades include U.S. Pat. No. 6,615,496 B1 entitled Micro-machined Cutting-blade Formed From {211} Oriented Silicone and assigned to Sandia Corporation. Another example of a ceramic cutting-blade is disclosed in U.S. Patent Application Number U.S. 2003/0199165 A1 entitled System and Method For the Manufacture of Surgical Blades and assigned to Becton, Dickinson, and Company. These ceramic blades promise more precise blade extension and cleaner and shaper cutting edges than those achievable from the use of razor blade stock.

Also, it is typically easier to manufacture a ceramic blade having a single-bevel cutting edge as opposed to the multi-bevel cutting edge of the razor blade stock.

It has come to be appreciated that the use of a single-bevel cutting-blade assembly in a microkeratome designed for a multi-bevel cutting-blade assembly requires special orientation that is not readily apparent. Therefore, it would be desirable to have a replacement cutting-head for a microkeratome which accommodates a single-bevel cutting-blade assembly but yet achieves similar corneal flap results compared to the use of a multi-bevel cutting-blade assembly formed from razor blade stock.

### BRIEF DESCRIPTION OF THE DRAWINGS

FIG. 1 is a side elevation view of a multi-bevel cutting-blade assembly;

FIG. 2 is an enlarged portion of FIG. 1;

FIG. 3 is a side elevation view of a cutting-blade assembly for a single-bevel cutting-blade assembly;

FIG. 4 is an enlarged portion of FIG. 3;

FIG. 5 is a partial view of a cutting-head including an orientation of both a multi-bevel cutting-blade and a single-bevel cutting-blade; and

FIG. 6 is a cutting-head with a single-bevel cutting-blade assembly in accordance with the present invention.

### DETAILED DESCRIPTION

FIG. 1 shows a multi-bevel cutting-blade assembly 10 which is well known in the prior art and includes a blade holder 12 and a cutting-blade 14 which is held within a cutting-head of a microkeratome, such as a Hansatome™ available from Bausch & Lomb Incorporated. Dashed circle 16 identifies the cutting edge portion of cutting-blade 14.

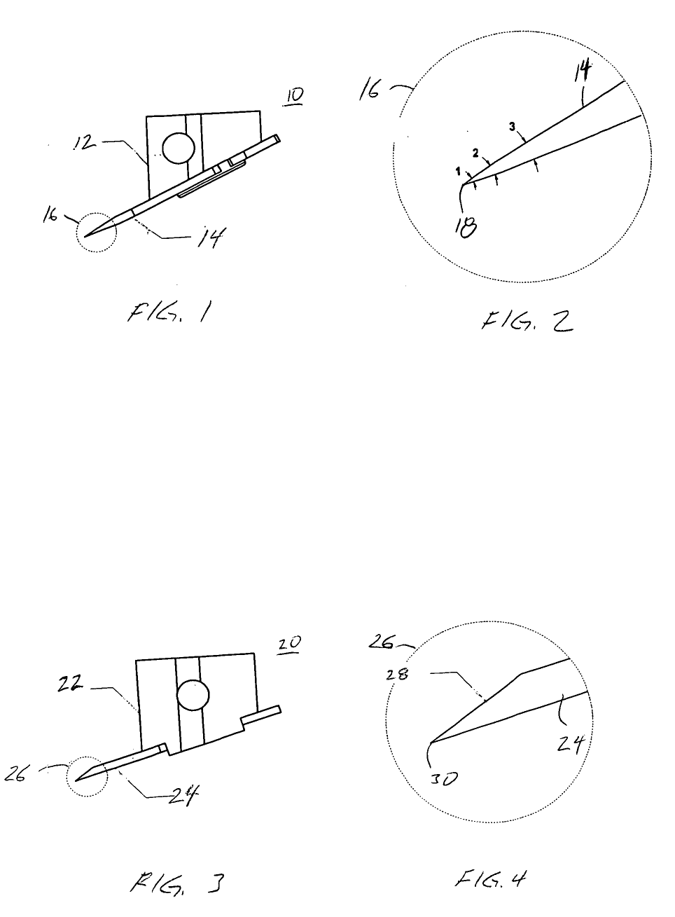

FIG. 2 is an enlarged section 16 showing a portion of cutting-blade 14 and identifies a typical cutting edge of cutting-blade 14 formed from razor blade stock. Numerals 1, 2, and 3 identify three different bevel angles formed on cutting-blade 14. This is typically found on razor blade stock, such as that available from American Safety Razor. And has been taught in the prior art, the cutting tip or actual edge 18 preferably is oriented relative to a cutting-head such that the corneal flap is not compressed between cutting tip 18 and a cutting-head (not shown).

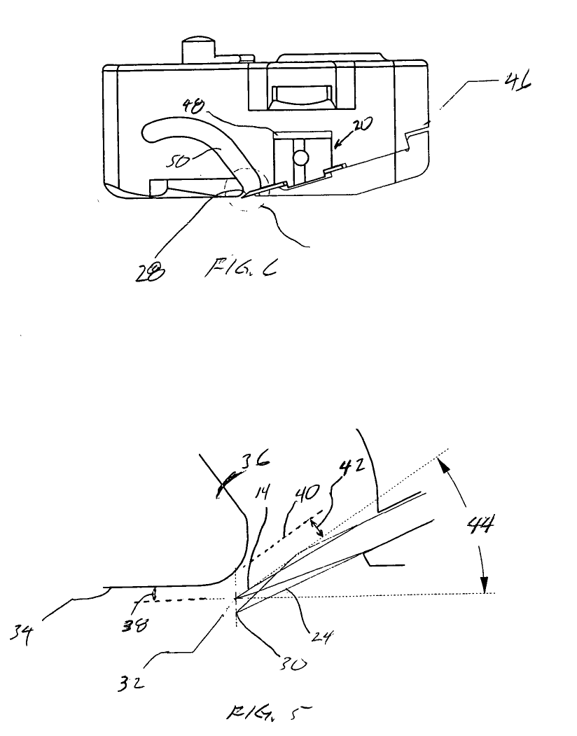

Recently, cutting-blade assemblies such as that shown in FIG. 3 have begun to be developed. Cutting-blade assembly 20 of FIG. 3 includes a holder 22, which is very similar to the holder 12 and is well known in the art. Holder 22 is typically formed from Delrin™ or molded from suitable plastic materials, such as Lubuloy™. Cutting-blade assembly 20 also includes a single-bevel cutting-blade 24 which may be formed from a number of different materials, including ceramic, metal, or plastic materials, such as PMMA (poly(methyl methacrylate)). For cutting-blades 24 formed from ceramic or other materials, it may be easiest to form a single-bevel cutting edge as shown within dashed line 26. FIG. 4 is an enlarged area 26 showing a single-beveled edge 28 and a tip 30. It has come to be appreciated that when placing a single-bevel cutting-blade in a cutting-head for a microkeratome originally designed for use with a multi-beveled cutting-blade assembly, that at least two factors need to be considered when orienting the single-bevel cutting-blade in a cutting-head.

First, the cutting tip 30 of the single-bevel cutting-blade needs to be placed at the blade-edge point 32 to achieve the same corneal flap thickness as that of the multi-beveled cutting-blade 14. Typically, for a 180 micron head, the blade-edge point 32 will be 0.0044 millimeters below the aplanation surface 34 of cutting-head 36 as identified by numeral 38. Also, blade-edge point 32 will be 0.0077 millimeters from tangent line 40 and shown at numeral 42. The distances 38 and 42, which define blade-edge point 32, ensure that the corneal flap will not be compressed by the cutting-head 36. However, it has come to be appreciated that by simply raising cutting tip 30 to blade-edge point 32 is insufficient to ensure that a clean, undamaged corneal flap is formed by a single-bevel blade 24. The multi-beveled blade 14 also includes a shear-face angle shown at 44 and is typically 35° for the Hansatome™ but could be other angles for other microkeratomes. The shear-face angle is formed along a line extending from the bevel angle 1 of FIG. 2. Bevel angle 28 of FIG. 4 should therefore be oriented to fall along and form the same shear-face angle 44 as that formed by the multi-bevel blade 14.

FIG. 6 shows a cutting-head 46 including a cutting-blade assembly 20 having a single-bevel cutting edge 28. Cutting-head 46 includes a slot 48 allowing cutting-blade assembly 20 to be oscillated as the cutting-head 46 is moved across a patient's eye (not shown). The single-bevel cutting-blade assembly 20 is oriented within the cutting-head 46, such that a shear-face angle and blade-edge point are each approximately the same as those for the multi-beveled cutting-blade assembly. By having the shear-face angle the same for the single-bevel blade assembly 20 as that for the multi-bevel blade assembly 10, it is ensured that the corneal flap is not compressed after the corneal tissue has been cut and is being directed into the corneal flap recess 50. This ensures that the corneal flap that is formed will not be damaged by being compressed between the single-beveled cutting-blade and the cutting-head. A replacement cutting-head 46 includes a cutting-blade assembly 20 having a single-bevel cutting edge 28. The cutting-head includes a slot 48 that allows the cutting-blade assembly 20 to be oscillated as the cutting-head 46 is moved across a patient's eye. The single-bevel cutting-blade assembly 20 is oriented within the cutting-head 46, such that a shear-face angle 44 and a blade-edge point 32 are each approximately the same as those for the multi-bevel cutting-blade assembly 10.

The single-bevel blade 24 has been shown to have the beveled edge 28 oriented on the top side or nearest the holder 22. As those skilled in the art will realize, it is possible to orient the beveled edge 28 on the opposite side without departing from the scope of the present invention.

1. A replacement cutting-head for a microkeratome, wherein an original cutting-head was designed for use with a multi-beveled cutting-blade assembly having a known shear-face angle and a blade-edge point, the replacement cutting-head comprising:
a cutting-blade assembly having a single-bevel cutting edge;
a cutting-head including a slot allowing the cutting-blade assembly to be oscillated as the cutting-head is moved across a patient's eye; and
wherein the single-bevel blade assembly is oriented within the cutting-head, such that a shear-face angle and blade-edge point are each approximately the same as those for the multi-beveled cutting-blade assembly.

2. The cutting-head of claim 1, wherein the single-bevel cutting-blade has a shear-face angle of about 35° and a blade-edge point about equal to a desired corneal flap thickness.

3. A cutting-head for a microkeratome comprising:
a cutting-head including a slot for holding a single-bevel cutting-blade assembly, such that the cutting-blade assembly is oscillated as the cutting-head is moved across a patient's eye; and
wherein the slot is oriented, such that the single-bevel cutting-blade assembly will be positioned to have a shear-face angle and a blade-edge point to provide a known corneal flap thickness without compressing the corneal flap.

4. The cutting-head of claim 3, wherein the single-bevel cutting-blade has a shear-face angle of about 35° and a blade-edge point about equal to a desired corneal flap thickness.

5. A replacement cutting-head for a microkeratome, wherein an original cutting-head was designed for use with a multi-beveled cutting-blade assembly having a known shear-face angle and a blade-edge point, the replacement cutting-head comprising:
a cutting-head including a slot for holding a single-bevel cutting-blade assembly, such that the cutting-blade assembly may be oscillated as the cutting-head is moved across a patient's eye; and
wherein the slot is oriented, such that the single-bevel cutting-blade assembly will be positioned to have a shear-face angle and a blade-edge point each approximately the same as those for the multi-beveled cutting-blade assembly.

6. The cutting-head of claim 5, wherein the single-bevel cutting-blade has a shear-face angle of about 35° and a blade-edge point about equal to a desired corneal flap thickness.
